(19) United States
(12) Patent Application Publication
(10) Pub. No.: US 2021/0322695 A1
(43) Pub. Date: Oct. 21, 2021

(54) Method and Device for Assisting and Enforcing a breathing process

(71) Applicants: Mikhail Levitin, Reeders, PA (US); Lev D. Kucher, Brooklyn, NY (US)

(72) Inventors: Mikhail Levitin, Reeders, PA (US); Lev D. Kucher, Brooklyn, NY (US)

(21) Appl. No.: 16/852,492

(22) Filed: Apr. 19, 2020

(51) Int. Cl. A61M 16/00; A61M 16/20; A61M 16/10; A61H 31/00; A61H 9/00; A61M 16/06

(52) U.S. Cl. CPC A61M 16/022; A61H 2201/107; A61M 16/105; A61M 16/1075; A61M 16/0081; A61H 31/00; A61H 9/0092; A61M 16/06; A61M 2205/8275; A61M 2202/0208; A61M 2230/432; A61M 2230/435; A61H 2201/1238; A61M 2205/07; A61M 16/201

(57) ABSTRACT

The present invention relates to the art of automatic regulation of pulmonary devices for assisting and/or enforcing the breathing process by converting Bag-Valve-Mask (BVM) apparatus are also known as manual resuscitators to automatic system by pneumatic matter with the goal to enhance both phases of breathing: inhalation and exhalation. It also replaces a mechanical chest compression for automatic pneumatic compression, could be complimented with the use of the TENs unit and can be used for extended periods of time with a high level of reliability, simplicity, efficacy and low cost.

This portable and light device is recommended to be used as a resuscitator attached to the patients with mild to extremely suppressed or without respiratory drive.

The source of power can be electrical, battery operated, manual or a combination thereof.

That feature is extremely critical for its use in a combat zone or during a power failure.

### BACKGROUND OF THE INVENTION

A breathing process consists of two stages. The first stage of breathing, inhaling air into your lungs, is called inspiration (I) or inhalation. Inspiration happens because of a large breathing muscle called the diaphragm and muscles between your ribs contract, creating a negative pressure or vacuum inside the chest cavity.

When you take a breath, the air goes in through your nose and mouth and travels down your lungs (the first stage of a breathing cycle). The negative pressure draws the air that you breathe into your lungs.

The second stage of breathing, blowing air out of the lungs, is called expiration (E) or exhalation. After the oxygen and carbon dioxide trade places in the alveoli, the diaphragm relaxes and positive pressure is restored to the chest cavity. This forces the used air out of the lungs, following the reverse of the path that it used to get in the lungs. The entire breathing process is repeated 10 to 30 times per minute depend on the condition of the person.

Our device is designed to imitate the same physiological process by converting mechanical BVM into automatic one.

Inspiratory time and expiratory time are then determined by portioning the respiratory cycle based on the set ratio. For instance, a patient with a respiratory rate of 10 breaths per minute will have a breath cycle lasting 6 seconds. A typical I:E ratio for most situations would be 1:2, if we apply this ratio to the patient above, the 6-second breath cycle will break down to 2 seconds of inspiration and 4 seconds of expiration. Increasing the I:E ratio to 1:3 will result in 1.5 seconds of inspiration and 4.5 seconds of expiration. Thus, a “higher” I:E ratio results in less inspiratory time and more expiratory time in the same length of the breath cycle. In addition, we can regulate the I:E ratio with the power of the air flow during inspiratory and expiratory time.

Patients with lung failure are often prescribed mechanical ventilation as a lifesaving intervention while waiting the recovery of the patient's own lungs, or as a bridge to lung transplantation. The currently available technology for ECMO or for high frequency ventilators requires large very bulky and expensive machines that practically have a selective usage. The large, heavy nature of these machines is not user friendly as well. These devices are not truly portable and require the assistance of another person, often the presence of trained, licensed specialists to be physically available around the clock to ensure that these systems are functioning properly or is involved the administrative physical activities that has a limit due to exhaustion.

The need for near constant supervision by a specialist greatly reduces their ability to spend time with multiple patients that is critical during an intensive care.

Hospitals across the US and around the world brace for cases of the novel coronavirus, departments in charge of caring for the sickest patients are grappling with how they are going to respond.

By some estimates, millions of Americans sickened by coronavirus might need a stay in an intensive care unit, the part of the hospital devoted to providing advanced life-saving care. That will likely put a strain on staff, supplies of equipment like ventilators and put facilities at enormously high mortality risk.

Although invasive mechanical ventilation saves tens of thousands of lives each year, it can also be harmful, causing or worsening acute respiratory distress syndrome (ARDS) when misapplied. The repetitive stretching of lung tissue during positive pressure ventilation can damage fragile alveoli already made vulnerable by pre-existing illness. This potentially lethal process has been called ventilator-induced lung injury. That is why the emphasis should be moved on the second stage, exhalation, to remove excessive amount of CO₂. If carbon dioxide levels are allowed to accumulate without disposal, the blood will become more acidic, leading to cellular damage on a systemic scale, which may ultimately lead to organ failure or death.

The patients with COVID-19 often have what seems to be a pervasive but initially overlooked feature of “silent hypoxia”. Unlike many other respiratory diseases, COVID-19 can slowly starve the body of oxygen without initially causing much shortness of breath. By the time some patients have trouble breathing or feel pressure in the chest, among the symptoms the U.S. Centers for Disease Control and Prevention lists as emergency warning signs, they are already in life threaten condition.

Thus, there is a dramatic need in medicine for a portable, reliable, simple and inexpensive integrated breathing assisting automatic device which does not require specially trained personnel to operate it and can also help by releasing medical personnel from constant patient supervision for additional vital tasks.

Further, there is a need for such a device that can imitate physiological breathing stages, does not require significant financial investment, especially in critical ambulance situations, while highly increasing the number of patients that requested a critical care, eventually saving more lives. The simplicity, low-cost and ease of production of said device, and the fact that it can be made within a short time ought to put it in great demand in any health care system. The present invention addresses this unmet need in medicine.

### SUMMARY OF THE INVENTION

In one embodiment, a portable device for assisting and/or enforcing breathing process, includes a compressor with negative air pressure, having a filter to disinfect the air coming from a pipe connected to the inlet of a monitor with internal distributor which regulates time and pressure of discharging portions of the air exhaled from the patient through a BVM, which operated in a durable pressure chamber, and into a monitor, where the regulation is achieved according to a O₂ censor and by certain speed of said distributor and certain size of bilateral adjustable windows on said distributor through an outlet from the nose and/or mouth of the patient with extremely suppressed or without respiratory drive.

More specifically, a preferred embodiment of the present invention includes an engine controlled distributor which regulates a positive or a negative pressure supply to durable pressure chamber with BVM that allow the air to flow from the patient with mild to extremely suppressed or even without respiratory drive to imitate natural physiological breathing cycles.

According to the second embodiment of the present invention a compressor with positive pressure provides air flow through the monitor to the BMV inside durable pressure chamber, deflates the bag (BVM) and sends the air from said bag to the nose and/or mouth of the patient with extremely suppressed or without respiratory drive, and said energy support system feeds said compressor and said distributor's engine which regulates frequency, amount of flow and depth of the patient's breathing.

According to the third embodiment of the present invention a positive pressure from said compressor affects said rigid durable pressure chamber and without a physical contact with the ball compresses BVM forcing the air through an inlet to flow from BVM to the patient to initiate an inhalation action while a positive pressure valve is opened and a negative pressure valve is closed. In turn, when a positive pressure is off said positive pressure valve is closed and the negative pressure valve is opened and negative pressure inside said durable pressure chamber remains constant or variable and through an outlet engages an exhalation process as a result of expending of said ball of said BVM. When a positive pressure exceeds the maximum preset level or negative pressure plunges below minimum preset level a control valve temporally opens to normalize the pressure inside the pressure chamber to the preset comfort pressure zone.

According to the fourth embodiment of the present invention a combination of the first and second embodiment is set up as one process (inhalation) follows another (exhalation) in repetitive cycling motions.

According to the fifth embodiment of the present invention the air supply provided with a compressor with modulated parameters of air flow according to the results of the O₂/CO₂ sensor and affects the pressure in the rigid durable pressure chamber without a physical touch compresses BVM forcing the air from said ball to the nose and/or mouth of the patients who has from mild to extremely suppressed or no respiratory drive.

According to the sixth embodiment of the present invention the air flow alternated by gas from a therapeutic gas chamber to said compressor through said pipe to an inlet of said monitor passing outlet through thermo unit affecting a pressure in said rigid durable pressure chamber and without a physical contact compresses BVM forcing the air through an inlet to flow from BVM to the nose and/or mouth of the patients according to the results of the O₂/CO₂ sensor.

According to the seventh embodiment of the present invention the compressor produces an air flow that pushes an upper valve in the pump in closing position and presses movable membrane against spring while air flow is produced by said membrane which closes on its way open valves and delivers air through the outlet to nose and/or mouth of said patient. When air pressure from said compressor stops, the upper valve gets open by gravity and by the force of said membrane which is pushed back by it's spring, empting the part of the pump from air into opened outlet and at the same time sucking air from said patient and supplying inlet into opened outlet.

According to the eighth embodiment of the present invention the air flow from said compressor with preset level of positive/negative pressure air connected through said pipe to an inlet of said monitor which regulates time and pressure of supplying portions of the air from outlet through a pipe to a chest cuff on the patient's chest to accommodate the exhalation phase with the additional compression by the chest cuff and/or this process complemented with TENs like unit.

According to the ninth embodiment of the invention the all functions will be the same as the first one except the monitor and regulator would be replaced for a solenoid with the same functions.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is an illustration of the system that provides a positive or a negative pressure inside the durable pressure chamber with BVM, which affects the air flow in the mask attached to a nose and/or mouth of the person according to one embodiment.

FIG. 2 is a system diagram of the device according to the second embodiment.

FIG. 3 is a system diagram of the device according to the third embodiment.

FIG. 4 is a system diagram of the device according to the fourth embodiment.

FIG. 5 is a system diagram of the device according to the fifth embodiment.

FIG. 6 is a diagram with a therapeutic gas chamber according to an exemplary embodiment of the device.

FIG. 7 is a diagram with the independent pump and membrane to regulate an inspiration and expiration of a patient according to an exemplary embodiment of the device.

FIG. 8 is a system diagram of the device complimented with a chest cuff according to the eighth embodiment.

FIG. 9 is a system diagram of the device reequipped with a solenoid according to the ninth embodiment.

### DETAILED DESCRIPTION

It is to be understood that the figures and descriptions of the present invention have been simplified to illustrate elements that are relevant for a clear understanding of the present invention, while eliminating, for the purpose of clarity, many other elements found in the field of artificial lungs or lung assist devices for individuals without respiratory drive. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which the invention pertains. Although any methods and materials similar or equivalent to those described herein can be used in testing the present invention, the preferred materials and methods are described herein.

It is also to be understood that the terminology used herein is for the purpose of describing particular embodiments only, and is not intended to be limiting.

The terms “patient,” “subject,” “individual,” and the like are used interchangeably herein, and refer to any animal amenable to the systems, devices, and methods described herein. Preferably, the patient, subject or individual is a mammal, and more preferably, a human. The description of “positive” pressure means that it is above the ambient one “negative” pressure means below the ambient pressure.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The present invention relates to the art of automatic pneumatic regulation of pulmonary devices for assisting and/or enforcing the breathing process which can be used for extended periods of time with a high level of reliability, simplicity and efficacy not by a specially trained technician, but by volunteers, family members or by a patient.

The following is a detailed description of the best presently known mode of carrying out said invention. This description is not to be taken in a limiting sense, but is made merely for the purpose of illustrating the general principles of the invention. The scope of the invention is defined by the appended claims.

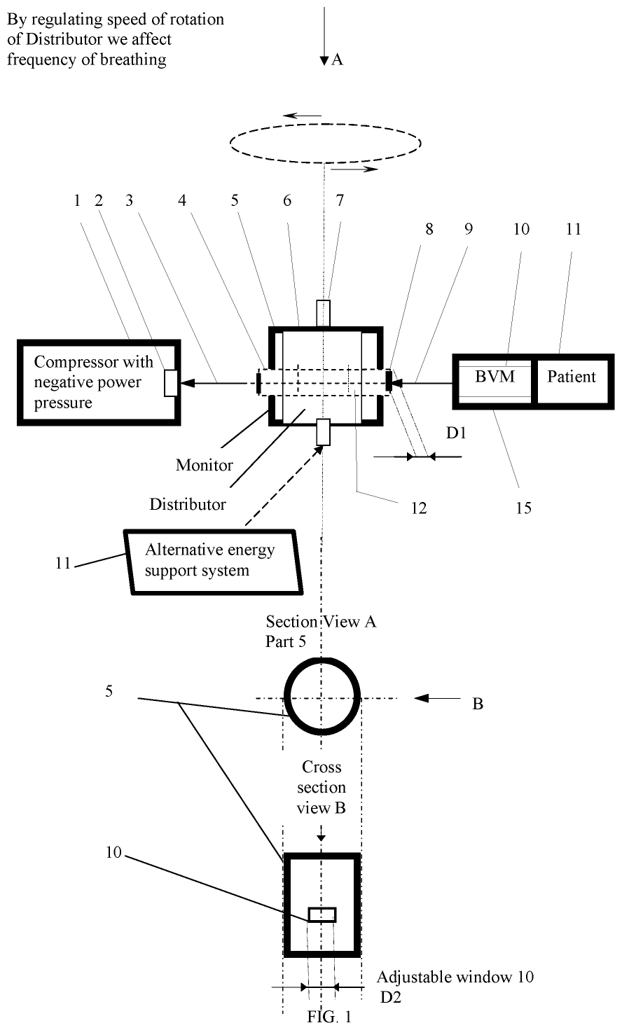

Referring to FIG. 1 the air flow Compressor 1 with negative air pressure, having a filter 2 to disinfect the air coming from pipe 3 connected to the inlet 4 of said monitor 5 with internal rotating distributor 6 which regulates time and pressure of discharging portions of the air exhaled from the patient 11 through said BVM 10, which operated in said durable pressure chamber 15 under constant or variable level of negative pressure, and into said monitor 5, where the regulation is achieved according to said O₂/CO₂ censor 13 and by certain speed of said distributor 6 and certain size of said bilateral adjustable windows 12 on said distributor 6 through the outlet 8 from the nose and/or mouth of the patient 11 with a mild to extremely suppressed or without respiratory drive.

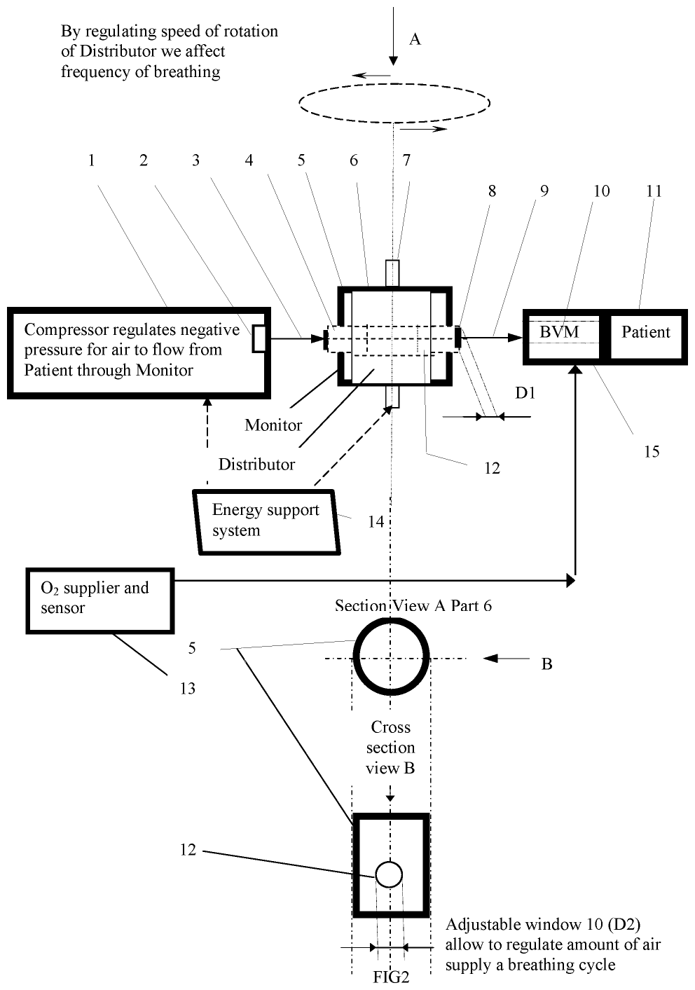

Referring to FIG. 2 the air flow from said compressor 1 through said pipe 3 with preset constant or variable level of compressed air connected to an inlet 4 of said monitor 5 with said distributor 6 that regulates time and pressure of supplying portions of the air/gas from outlet 8 through pipe 9 to the BVM 10, which operated in said durable pressure chamber 15 under positive pressure with a constant or variable level directed to the patient 11, where the regulation provided according to a said O₂/CO₂ censor 13 and by said distributor 6 and said shift 7 fed by an energy support system 14 and by certain size of bilateral adjustable windows 12 on said distributor 6.

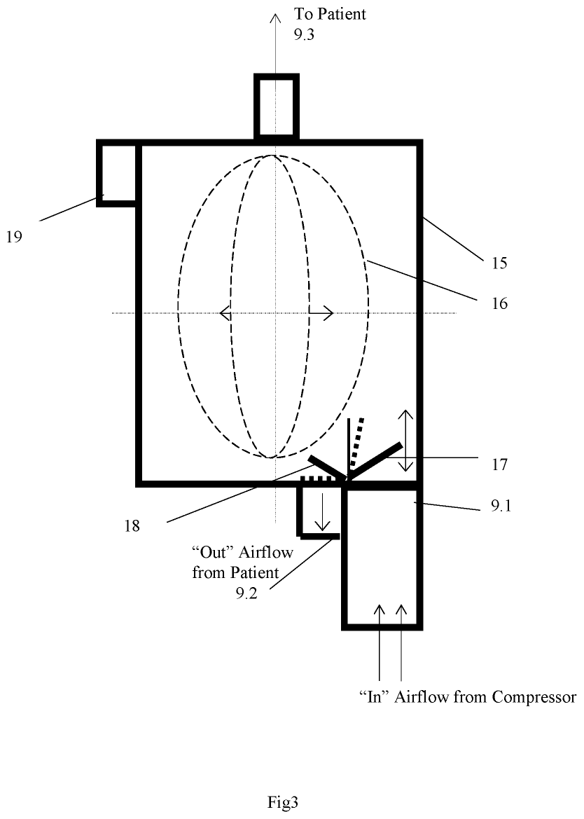

Referring to FIG. 3 the air supply with a positive pressure enters rigid durable pressure chamber 15 through inlet 9.1 to make a compression of a flexible ball 16 of BVM 10 inside said chamber 15 without a mechanical contact and forcing the air flow from BVM 10 to the patient 11 to initiate an inhalation action while valve 17 is open and valve 18 is closed. In turn, when a positive pressure is off valve 17 is closed and valve 18 is opened a negative pressure through outlet 9.2 engaged an exhalation as a result of expending of said ball 16 of said BVM. When a positive pressure exceeds the maximum preset level or negative pressure plunges below minimum preset level a control valve 19 temporally opens to normalize the pressure inside the pressure chamber 15 within preset comfort pressure zone.

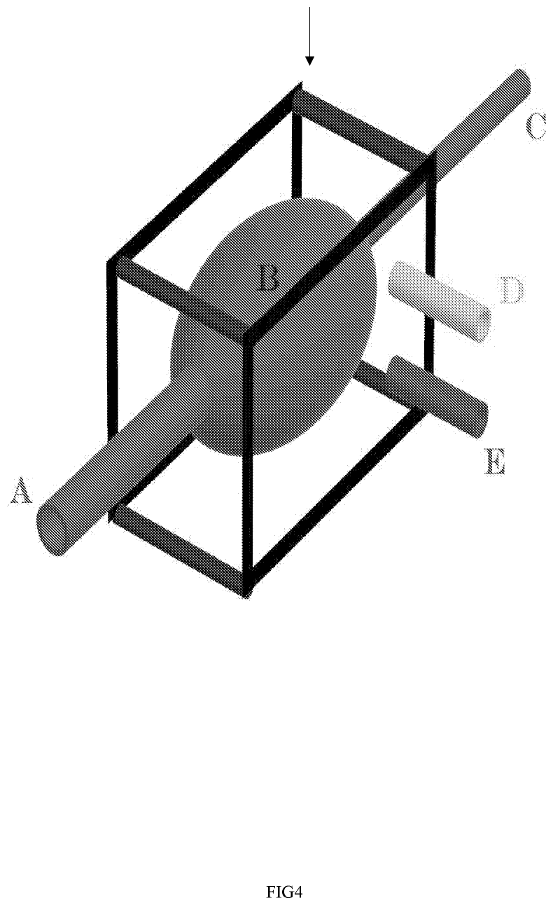

Referring to FIG. 4 a combination of the first and second embodiment is set up as one process, inhalation, followed by another exhalation in repetitive cycling motions where ABC air circulation line depicts the position of said BVM 10 in said firm durable pressure chamber 15 which receives a positive pressure from Line D and negative pressure from Line E to simulate a breathing cycle for the patient by manipulating said flexible ball 16.

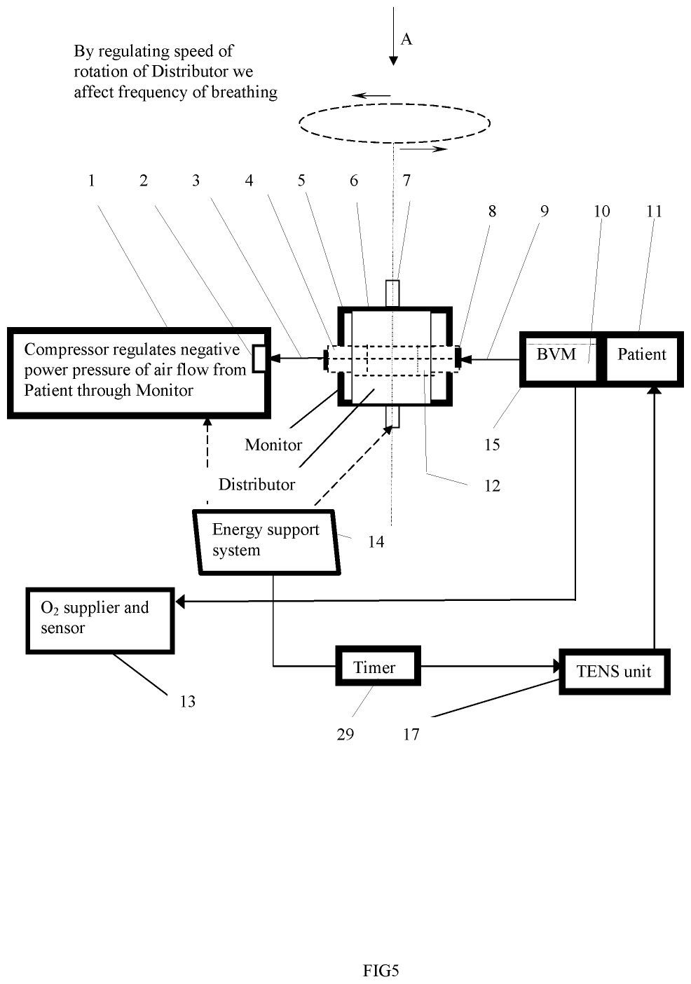

Referring to FIG. 5 the air flow compressor 1 with negative air pressure, having a filter 2 to disinfect the air coming from pipe 3 connected to the inlet 4 of said monitor 5 with said distributor 6 which regulates time and pressure of discharging portions of the air exhaled from the patient 11 through said BVM 10, which operated in said rigid durable pressure chamber 15, and into said monitor 5, where the regulation is achieved according to said O₂/CO censor 13 and by certain speed of said distributor 6 and certain size of said bilateral adjustable windows 12 on said distributor 6 through the outlet 8 from the nose and/or mouth of said patient 11 who's exhalation process is stimulated by attached to the patient's body TENS unit 17 controlled by a timer 29.

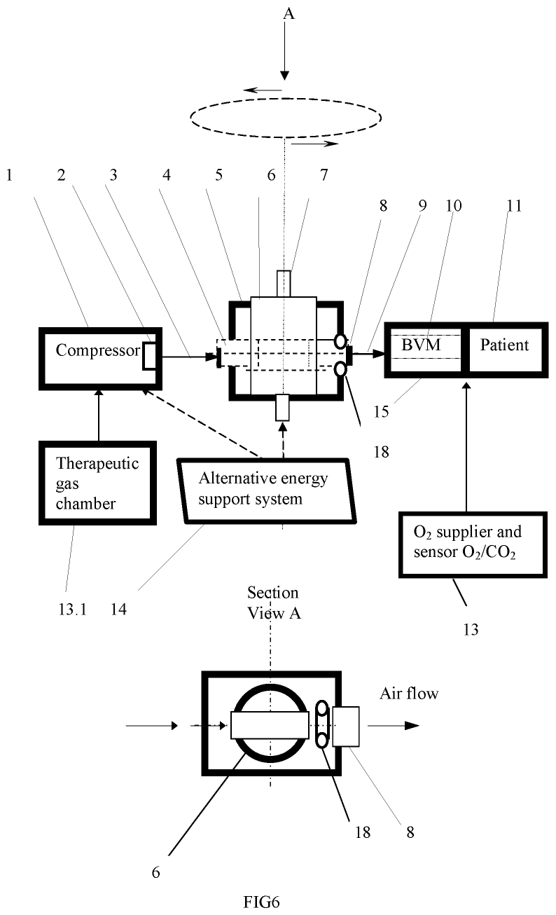

Referring to FIG. 6 the air flow alternated by therapeutic gas from a chamber 13.1 to said compressor 1 through said pipe 3 to an inlet 4 of said monitor 5 passing outlet 8 through thermo unit 18 following through the adjustable windows 12 to the nose and/or mouth of the patient's 11 mask through said BVM 10, which operated in said rigid durable pressure chamber 15, and said mask is supplied by O₂ and censored for a proper setting by said O₂/CO₂ censor 13.

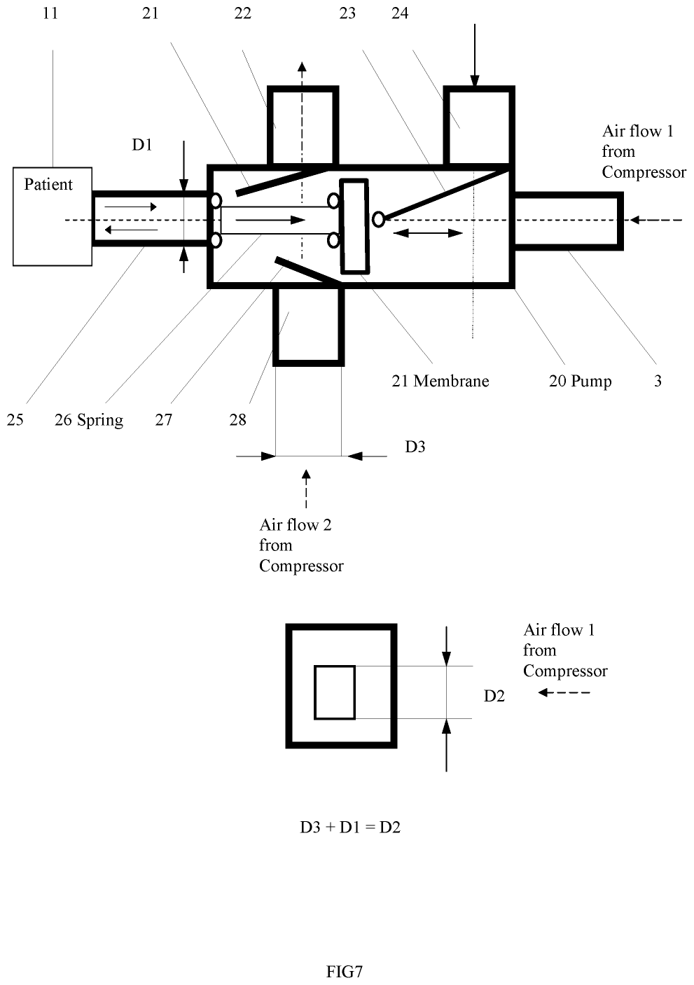

Referring to FIG. 7 the compressor 1 produces an air flow through an inlet 3 into a pump 20 where it moves a valve 23 in the pump 20 in closing position and presses movable membrane 21 against spring 25 while air flow produced by said membrane 21 and it closes on its way prior opened valve 21 and 27 and delivers air through the outlet 25 to nose and/or mouth of said patient 11. It imitated an inhalation phase of the breathing. When air pressure from said compressor 1 stops, the upper valve 23 gets open by gravity and by the force of the spring 26 of said membrane 21 which is pushed back by said spring 26, empting the part of the pump 20 from air into opened outlet 24 and at the same time sucking in air from said patient 11 and supplying inlet 28 into opened outlet 24. It imitates an exhalation phase of the breathing. The dimension D3+D1=D2 where the adjustable membranes on the outlets 3, 25 and 28 regulate the duration and ratio of the inspiration (I) and expiration (E) phases of the breathing process and I/E ratio to the right or to the left of this equation.

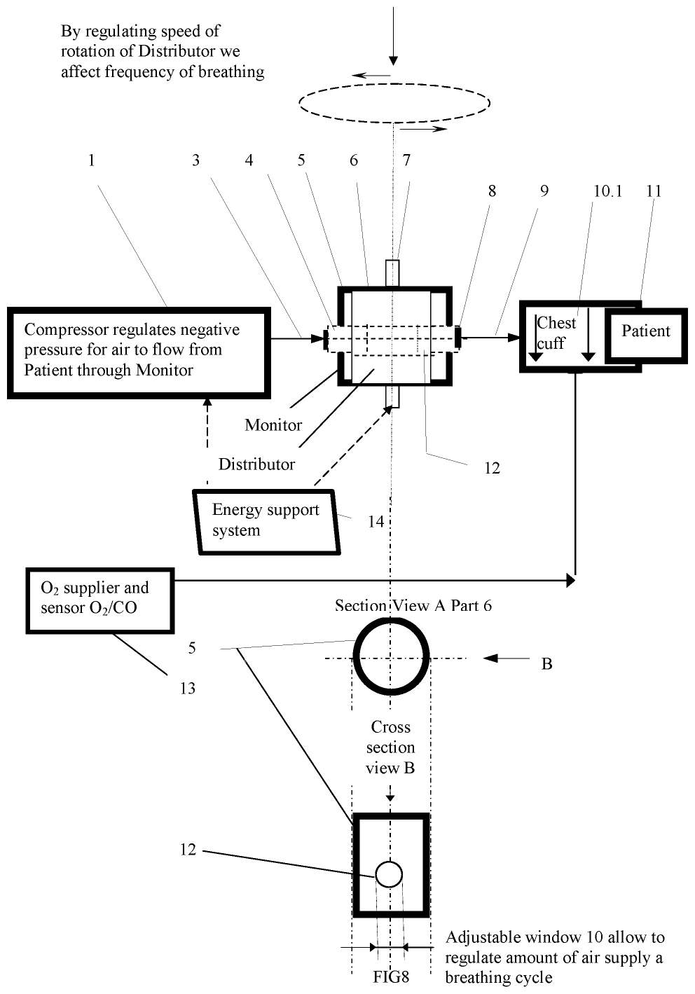

Referring to FIG. 8 the air flow from said compressor 1 with preset level of positive/negative constant or variable pressure air connected through said pipe 3 to an inlet 4 of said monitor 5 with said distributor 6 that regulates time and pressure of supplying portions of the air from outlet 8 through pipe 9 to a chest cuff 10.1 on the patient's 11 chest. The parameters are according to a O₂/CO₂ censor 13 and by said distributor 6 and said shift 7 fed by an energy support system 14 and by certain size of bilateral adjustable windows 12 on said distributor 6.

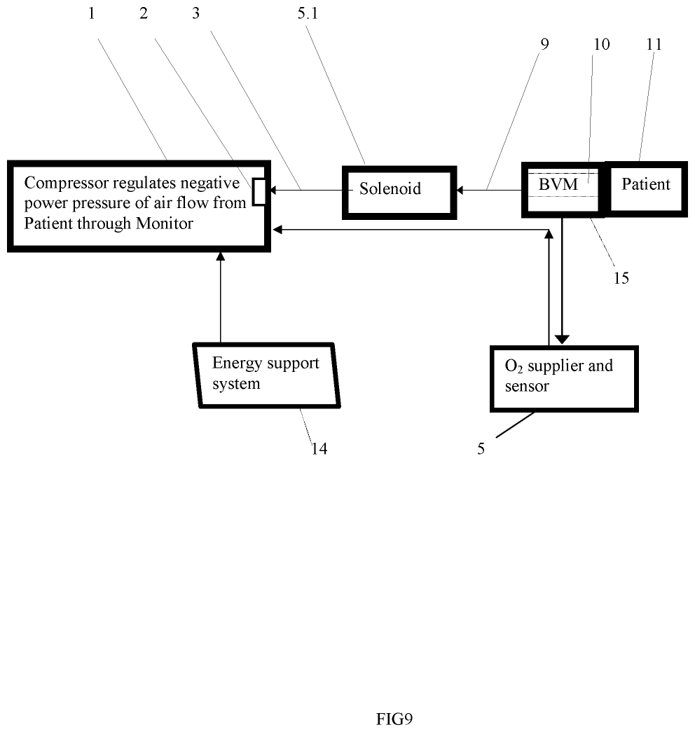

Referring to FIG. 9 the air flow compressor 1 with negative air pressure, having a filter 2 to disinfect the air coming from pipe 3 connected to the inlet 4 of said monitor 5 and said distributor 6 which regulates time and pressure of discharging portions of the air exhaled from the patient 11 through said BVM 10, which operates in said rigid durable pressure chamber 15, and said solenoid 5.1 regulates air flow through the pipe 9 from the nose and/or mouth of the patient 11 and/or air flow from said chest cuff 10.1.

In one embodiment of the present invention, ambient air, i.e., air in the environment immediately surrounding the device is used as the sweep gas feed to said device. Ambient air typically comprises 20.95% oxygen and less than 0.04% carbon dioxide by volume. By using ambient air as the gas feed for the device of the present invention eliminates the need for an oxygen tank or other source of oxygen, thereby increasing the portability of the device. Alternatively, in another embodiment, ambient air can be mixed with oxygen from an oxygen source prior to be supplied to the oxygenator in order to increase the concentration of oxygen in the gas feed, thereby increasing the rate of oxygen transfer to the blood.

In various embodiments, the device of the present invention may comprise additional components that will improve the performance of blood oxygenation and lung assistance. Such components include, but are not limited to: a thermo unit, humidifier, therapeutic gas, at least one sensor, an air filter, and a control panel or other means for controlling the device.

In one embodiment, the device of the present invention may comprise a thermo unit. In such an embodiment, the thermo unit is used to maintain the temperature of the air supply at or close to the subject's natural body temperature in order to prevent or reduce the potential for causing adverse health effects or for a medically supervised increase in core temperature for the purposes of eradicating viral colonies/clusters.

In various embodiments, the device of the present invention comprises at least one sensor for measuring variables related to the operation of the device. In one embodiment, the device comprises an oxygen sensor for determining the level of oxygen in the subject's blood. In another embodiment, the device comprises a carbon dioxide sensor for determining the level of carbon dioxide in the subject's blood. In one embodiment, the oxygen and/or carbon dioxide sensors can be used for measuring the concentration of a gas in the blood entering the device, i.e., pre-oxygenation. In another embodiment, the oxygen and/or carbon dioxide sensors can be used for measuring the concentration of a gas in the blood returning to the patient, i.e., post-oxygenation. In one embodiment, the device comprises at least one sensor for determining the composition of oxygen and/or carbon dioxide in the sweep gas. In one embodiment, the device comprises at least one flow sensor for measuring the flow rate of blood at a desired location in the system. In one embodiment, the device comprises a flow sensor for measuring the flow rate of sweep gas in the oxygenator. In one embodiment, the device comprises temperature sensors for determining the temperature of the air at a desired location in the device, for example, the temperature of the inhaled air delivered to the patient.

In one embodiment, the device comprises an air filter for filtering particulates or other impurities from the gas being supplied to or out of the patient. In one embodiment, the filter is capable of filtering about 95% of particles that are 0.3 microns or larger.

In various embodiments, the device of the present invention may comprise means for controlling the device, for example, to control variables such as, but not limited to, the flow rate of air through the device, the composition of sweep gas, and the temperature of air flowing through the device. In one embodiment, the control means is a compact controller integrated with the device, comprising a touch screen or other means for entering and/or displaying data. In another embodiment, the control means may comprise a computer processor integrated with the device that can be controlled via a wireless connection to a computer that is not physically connected to the device.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A portable pneumatic device for converting a manually operated BVM into automatic system for assisting and/or enforcing breathing process through the nose and/or mouth of the patients with mild to extremely suppressed or without respiratory drive, comprising:
a compressor with negative pressure and connected to a filter and having a pipe that is attached to an inlet of a monitor with internal rotating distributor which regulates time and pressure of extracted portions of the air from a patient, where the regulation is achieved by constant or variable negative pressure inside a rigid durable container with said bag that expelling air through an outlet from the individual's nose and/or mouth to implement an expiration (E).

2. The device of claim 1, wherein the compressor with negative pressure encompassed by a compressor with a positive pressure that is having a pipe connected to an inlet of a monitor with internal rotating distributor which regulates time and pressure of delivered portions of the air to said patient through said bag, deflated by a positive pressure inside durable pressure chamber where the regulation is achieved by changing amount of air, the frequency of it delivery and duration of each compression to supply air to the individual's nose and/or mouth to implement an expiration (E) followed by an inspiration (I).

3. The device of claim 1, wherein the compressor with negative pressure encompassed by a compressor with a positive pressure that is having a pipe connected to an inlet of a monitor with internal rotating distributor which regulates time and pressure of delivered portions of the air to said chest cuff on the patient, where the regulation to the patient's chest is achieved by changing a pressure inside said chest cuff which tightly attached on the patient's chest to implement an expiration (E) followed by an inspiration (I) when the pressure from said chest cuff is released.

4. The device of claim 2, wherein the depth of expiration and inspiration phases restricted by said control valve.

5. The device of claim 2, wherein the breathing gas in the mask of BVM is pure oxygen or its combination with other therapeutic vapors to be directed to the nose and/or mouth of the patient from mild to extremely suppressed or without respiratory drive.

6. The device of claim 2, wherein said engines of said devices receive a power from a mechanical source, for example, a pedaling system.

7. The device of claim 2, further comprising an air or a liquid based filter for filtering the air/gas from or/and to the individual.

8. The device of claim 2, further comprising a thermo unit in front of the outlet of the monitor in the way that air will pass said unit on the way out during inhalation.

9. The device of claim 2, wherein said monitor and regulator of said devices are replaced with a solenoid serving the same functions.

10. A medical method for treating individuals from mild to extremely suppressed or without respiratory drive who can benefit from repeatedly applied negative-positive pressure in the thorax, the method comprising:
Implementing a negative pressure to induce an expiration (E) immediately followed by a positive pressure to induce an inspiration (I);
Implementing and modifying inverse ratio ventilation (IRV). This is achieved by modifying the inspiratory to expiratory (I:E) ratio, with the intention to increase oxygenation by increasing the mean airway pressure (MAP);
Implementing Volume Control and Pressure Control ventilation modes using I:E ratios of 1:2, and up as high as 1:6 in certain populations in these cases with expiratory phase is set longer than the inspiratory phase to more closely mimic normal physiologic breathing;
Implementing Inverse Ratio Ventilation using I:E ratios of 2:1, 3:1, 4:1, and so on, up as high as 10:1, with inspiratory times that exceed expiratory times.

11. The method of claim 10, further comprising a medical method for treating individuals from mild to extremely suppressed or without respiratory drive who can benefit from repeatedly applied positive pressure on the thorax, the method comprising:
delivering a positive pressure into the chest cuff applied on the person during his/her expiration phase;
deflating a pressure in the chest cuff applied on the person during his/her inspiration phase;

12. A medical method for treating individuals with extremely suppressed or without respiratory drive who can benefit from a negative pressure to the thorax complimenting by TENS unit or similar to it device operating with the same as TENS parameters and applied to individual's core muscles, the method comprising:
delivering electric signals through electrodes applied to the core muscles of the patient;
regulating the width, frequency and power of said signals.

12. A medical method for treating individuals from mild to extremely suppressed or without respiratory drive who can benefit from a BVM complimenting inflating and deflating the ball of said BVM through changing ambient pressure outside of the ball inside said rigid durable pressure chamber that contains said ball with no mechanical contact with the ball of said BVM.
